SEQUENCE LISTING (10 sequences)

SEQ ID NO: 1 - protein, 5 aa, Artificial sequence
  Synthetic peptide
GQTNT

SEQ ID NO: 2 - protein, 5 aa, Artificial sequence
  Synthetic peptide
SKKKK

SEQ ID NO: 3 - protein, 13 aa, Artificial sequence
  Synthetic peptide
GNNDESNISFKEK

SEQ ID NO: 4 - protein, 20 aa, Artificial sequence
  Synthetic peptide
GQTDNNSSQSQQPGSGTTNT

SEQ ID NO: 5 - protein, 6 aa, Artificial sequence
  Synthetic peptide
XGQTNT

SEQ ID NO: 6 - protein, 6 aa, Artificial sequence
  Synthetic peptide
XSKKKK

SEQ ID NO: 7 - protein, 14 aa, Artificial sequence
  Synthetic peptide
XGNNDESNISFKEK

SEQ ID NO: 8 - protein, 14 aa, Artificial sequence
  Synthetic peptide
XGNNDESNISFKEK

SEQ ID NO: 9 - protein, 21 aa, Artificial sequence
  Synthetic peptide
XGQTDNNSSQSQQPGSGTTNT

SEQ ID NO: 10 - protein, 14 aa, Artificial sequence
  Synthetic peptide
XGNNDESNISFKEK

(19) United States
(12) Patent Application Publication
(10) Pub. No.: US 2005/0192217 A1
(43) Pub. Date: Sep. 1, 2005

(54) Utilization of lipopeptides or lipoproteins in wound treatment and infection prophylaxis

(76) Inventors: Peter Muhlradt, Braunschweig (DE); Ursula Deiters, Braunschweig (DE)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 10/748,033

(22) Filed: Dec. 30, 2003

Related U.S. Application Data
(63) Continuation of application No. 09/716,778, filed on Nov. 20, 2000, now abandoned.
Application No. 09/716,778 is a continuation-in-part of application No. PCT/EP99/03436, filed on May 19, 1999.

(30) Foreign Application Priority Data
May 20, 1998 (DE) 198 22 820.1

(51) Int. Cl. A61K 38/17; C07K 14/775

(52) U.S. Cl. 514/12; 530/359

(57) ABSTRACT

The invention concerns a pharmaceutical preparation for the treatment of wounds in animals or humans containing or consisting of a lipopeptide or lipoprotein which carries at the N-terminals a dihydroxypropyl-cysteine group with two, optionally long-chain, fatty acids bonded via ester bonds.

### STATE OF THE ART

Five stages of wound healing can be described:
1. Coagulation of blood and release of mediators from thrombocytes (after a few minutes),
2. inflow of leukocytes, that is, initially granulocytes, later macrophages and lymphocytes (day 1-3),
3. multiplication of various cells, such as fibroblasts, endothelial and epithelial cells (day 3-7),
4. wound contraction (day 7-9), and
5. rebuilding of the scar tissue (up to one year).

The inflow of granulocytes in Phase 2 causes the uptake of debris and killing of infectious germs, a task which is also taken over by macrophages. Moreover, macrophages are the source of a number of mediators, such as signal peptides, growth factors and cytokines, such as the Transforming Growth Factor (TGFβ1), Platelet-Derived Growth Factor (PDGF-AA and BB), Fibroblast Growth Factor (FGF2) and TGF-α, which belongs to the family of the Epidermal Growth Factor (EGF). Macrophages also secrete interleukin-1 (IL-1), which induces FGF7 in the fibroblasts indirectly. All these factors participate in the different stages of wound healing and are indispensable for it. Without macrophages as a source for these, wound healing is highly delayed or is not possible.

### PROBLEMS

Although it is possible to use some of the mediators mentioned above in the isolated form during wound healing, this is ineffective since most of these peptides have a half-life of only a few minutes. Other difficulties consist in the fact that the natural point in time of the appearance of the different mediators, the optimal dosage and the interaction of these substances are not known in detail, and therefore could not be controlled during application.

A complication of surgical wounds, too, can be infections which generally lead to delay in wound healing and to an increase of the formation of scar tissue, which is problematic, especially in the case of cosmetic operations. Prophylactic coverage with antibiotics is not used any longer in many places, considering resistance problems and possible allergic reactions. In certain patient groups, for example, in diabetics or in elderly patients, wound healing is delayed.

### SOLUTION

Mycoplasmas naturally have the property of inducing the inflow of leukocytes at the location of infection, for example, in the lung. As we were able to show, this property is associated with the presence of a certain class of lipopeptides, which is characterized by the fact that they have an N-terminal with a dihydroxypropyl cysteine group to which two long-chain fatty acids are bonded via ester bonds [Mühlradt et al., J. Exp. Med., 185:1951-1958, (1997) and Mühlradt et al., Infect. Immun., 66:4804 (1998)]. In addition to the property already described, to stimulate murine as well as human macrophages and monocytes in vitro to liberate cytokines and prostaglandins (1 to 3; 11), these lipopeptides also have the ability, newly discovered by us, to induce in vivo a high titer of the chemokines MIP-1α, MIP-2, MCP-1 and KC and to cause the migration of leukocytes into the area [Deiters & Mühlradt, Infect. Immun., 67:3390 (1999)]. These lipopeptides stimulate the cells via the “toll-like receptor 2” [Takeuchi et al., J. Immunol., 164:554 (2000)].

In animal as well as human medicine, pure, synthetically produced lipopeptides, as well as lipopeptides that are encapsulated into liposomes or are coupled to biodegradable polymeric carriers can be used, for example, in the form of salves, lotions or injection solutions. When applied to wounds or injected near the wound, such preparations are supposed to increase and accelerate the natural inflow of granulocytes and macrophages and thus prevent infection as well as facilitate and accelerate wound healing by stimulation of the biosynthesis of mediators participating in wound healing, in the natural sequence and concentration. The in vivo effectiveness of such lipopeptides from mycoplasmas is surprising and new, since the application of other bacterial lipopeptides and their synthetic analogs do not show any effect in animal experiments [Hausschild et al., FEMS Immunol. Med. Microbiol., 8:77 (1994)].

### SUMMARY OF THE INVENTION

According to an embodiment, the problem underlying the invention is solved by the use of a lipopeptide or lipoprotein with the following general structure: 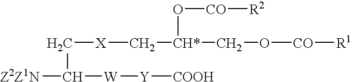
in which
R₁ and R², which can be the same or different, stand for C₇₋₂₅-alkyl, C₇₋₂₅-alkenyl or C₇₋₂₅-alkinyl,
X is S, O or CH₂,
Z¹ and Z², which can be the same or different, stand for H or methyl,
W stands for CO or S(O)ₙ (where n=1 or 2) and
Y stands for a physiologically compatible amino acid sequence consisting of 1 to 25 amino acid residues, and the asymmetric carbon atom marked with * has the absolute S-configuration when X═S (sulfur),
   for the preparation of a pharmaceutical preparation for animal or human wound treatment.

According to the invention, the lipopeptide or lipoprotein can be characterized by the fact that Y stands for a physiologically compatible amino acid sequence consisting of 1 to 25 amino acids.

According to the invention, the lipopeptide or lipoprotein can be characterized by the fact that Y stands for an amino acid sequence which is selected from the following group:

(i) aamino acid sequence, which does not have an adverse influence on the water solubility of the lipopeptide or lipoprotein.

| | | |
|---|---|---|
| (ii) | GQTNT | (SEQ ID NO:1) |
| (iii) | SKKKK | (SEQ ID NO:2) |
| (iv) | GNNDESNISFKEK | (SEQ ID NO:3) |
| (v) | GQTDNNSSQSQQPGSGTTNT | (SEQ ID NO:4) |

in the case of amino acid sequences (ii), (iii), (iv) and (v), individual amino acids may be absent or replaced.

According to the invention, the C₇₋₂₅-alkyl, C₇₋₂₅-alkenyl or C₇₋₂₅-alkinyl can be a C₁₅-alkyl, C₁₅-alkenyl or C₁₅-alkinyl.

According to the invention, in the C₇₋₂₅-alkenyl group, the double bond(s) have the cis-configuration.

According to another embodiment, the task on which the invention is based, to prepare a pharmaceutical preparation for the treatment of animal and human wounds, is solved by the use of a physiologically compatible lipopeptide or lipoprotein, in which the N-terminal carries a dihydroxypropyl cysteine group with two optionally long-chain fatty acids, which can be the same or different, bonded via ester bonds.

Furthermore, the invention concerns the use of a lipopeptide or lipoprotein, obtainable from a mycoplasma clone, for the preparation of a pharmaceutical preparation for animal or human wound treatment.

This application can be characterized by the fact that the lipopeptide or lipoprotein is obtainable from a Mycoplasma fermentans clone.

In the application according to the invention, the lipopeptide or lipoprotein can be water-soluble or amphoteric.

According to the invention, a lipopeptide or lipoprotein from the following group can be used:
(i) S-[2,3-bispalmitoyloxy-(2RS)-propyl]cysteinyl-GQTNT (SEQ ID NO:5)
(ii) S-[2,3-bispalmitoyloxy-(2RS)-propyl]cysteinyl-SKKKK (SEQ ID NO:6)

(iii) S-[2,3-bispalmitoyloxy-(2RS)-propyl]cysteinyl-

| | |
|---|---|
| GNNDESNISFKEK | (SEQ ID NO:7) |

(iv) S-[2,3-bispalmitoyloxy-(2S)-propyl]cysteinyl-

| | |
|---|---|
| GNNDESNISFKEK | (SEQ ID NO:8) |

(v) S-[2,3-bispalmitoyloxypropyl]cysteinyl-

| | |
|---|---|
| GQTDNNSSQSQQPGSGTTNT. | (SEQ ID NO:9) |

In the application according to the invention, the lipopeptide or lipoprotein can be used in the form of a solution for epicutaneous application, as an injection solution, as salve, as lotion, as aqueous suspension, a patch impregnated or coated with it, encapsulated in liposomes or coupled to biodegradable carrier polymers.

In the application according to the invention, the wounds can be wounds after injuries or surgical intervention, in order to treat chronically infected wounds, burn wounds, chronic ulcers or Ulcus venosum or wounds of patients who are corpulent or diabetic or were subjected to radiation or chemotherapy.

Synthetic lipopeptide preparations could be manufactured inexpensively and would enhance the natural progress of wound healing without causing any alteration in principle in the complicated regulatory mechanism of various mediators in wound healing. Moreover, infection prophylaxis would be achieved without having to use of antibiotics or other bacteriostatics that, for example, would affect wound healing. Such lipopeptide preparations could also be used for wounds on the face, where, for reasons of toxicity, the usual bacteriostatics are forbidden, when there is a danger of contact with the eyes or the nasal and oral regions. In topical applications, the danger of systemic effects, such as fever, is practically completely excluded.

The invention will be explained below in more detail with the aid of figures and examples.

### BRIEF DESCRIPTION OF THE DRAWINGS

FIG. 1A: The macrophage-stimulation activity of MALP-A:S-[2,3-Bispalmitoyloxy-(2RS)-propyl]cysteinyl-SKKKK (SEQ ID NO:6).

FIG. 1B: The macrophage-stimulation activity of N-Palmitoyloxy-MALP-A:S-[2,3-Bispalmitoyloxy-(2RS)-propyl]-N-palmitoyl-cysteinyl-SKKKK (SEQ ID NO:6).

FIG. 1C: The macrophage-stimulation activity of MALP-H: S-[2,3-Bispalmitoyloxy-(2RS)-propyl]-cysteinyl-GQTNT (SEQ ID NO:5).

FIG. 1D: The macrophage-stimulation activity of S-MALP-2: S-[2,3-Bispalmitoyloxy-(2S)-propyl]-cysteinyl-GNNDESNISFKEK (SEQ ID NO:8); and R-MALP-2: S-[2,3-Bispalmitoyloxy-(2R)-propyl]cysteinyl-GNNDESNISFKEK (SEQ ID NO:10).

FIG. 2 shows the inflow of the total leukocytes and granulocytes as a reaction to the intraperitoneal injection of 9 μg of racemic MALP-2 into NMRI mice (groups of 6 animals).

FIG. 3 shows the inflow of the total leukocytes and granulocytes as a reaction to the intraperitoneal injection of 0.2 mg of liposomes, which contained 9 μg of racemic MALP-2, in NMRI mice (groups of 6 animals).

FIG. 4 shows the inflow of total leukocytes and granulocytes as a reaction to the intraperitoneal injection of 0.2 mg of control liposomes which contained no MALP-2, in NMRI mice (groups of 6 animals).

FIG. 5A shows the infiltration of leukocytes in the skin on the back of an NMRI mouse, 3 days after intracutaneous injection of 2 μg of S-MALP-2, which was incorporated into liposomes.

FIG. 5B shows, in comparison: the skin on the back of an untreated mouse.

FIG. 6 shows the area of new tissue with new vessels (middle of the picture) formed after MALP injection.

FIG. 7 shows a significant acceleration of wound healing in the case of the animals treated with S-MALP-2.

### DETAILED DESCRIPTION OF PREFERRED EMBODIMENTS OF THE INVENTION

### EXAMPLE 1

Macrophage Activation by Synthetic Lipopeptides Derived from Mycoplasmas, Measured with the Nitrogen Monoxide Liberation Assay

An easily quantifiable assay for the activation of murine macrophages with lipopeptides is the liberation of nitrogen monoxide from peritoneal exudate cells in the presence of interferon-γ; see, for example, Mühlradt & Frisch, Infect. Immun., 62:3801-3807 (1994). This assay is carried out with peritoneal exudate cells of C3H/HeJ mice, which react only slightly with endotoxin, using microtiter plates (96 wells). Simultaneously, 10⁵ cells are stimulated with recombinant IFN-γ and a serial dilution of a material which activates macrophages. After incubation for 48 hours, the nitrate is reduced with nitrate reductase; NO is determined as the sum of nitrite and nitrate with the Griess reagent.

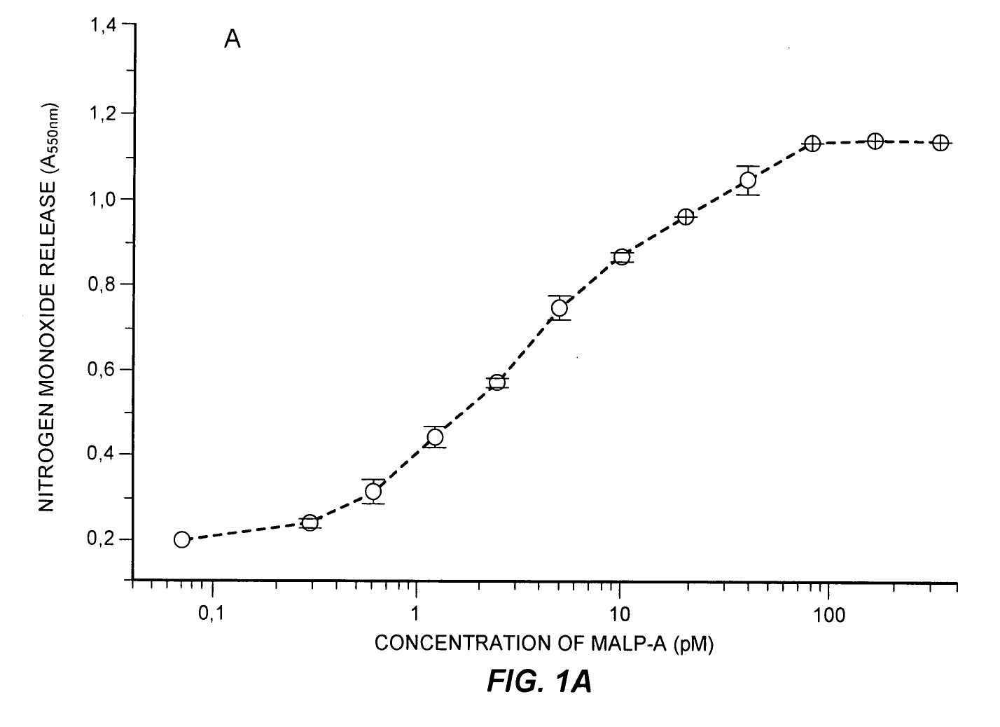

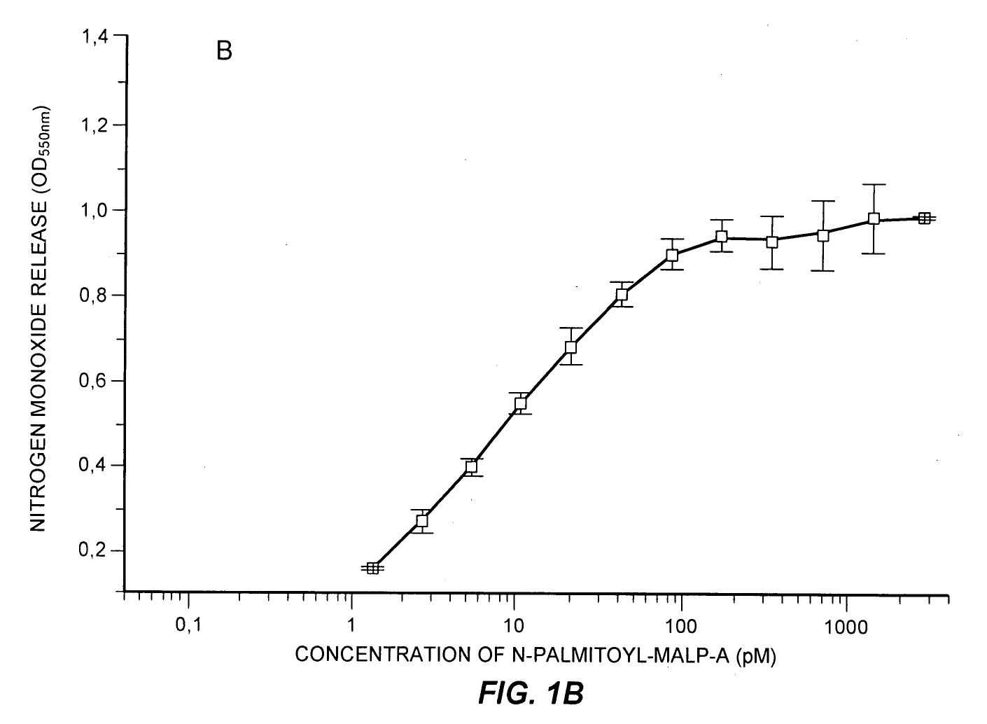

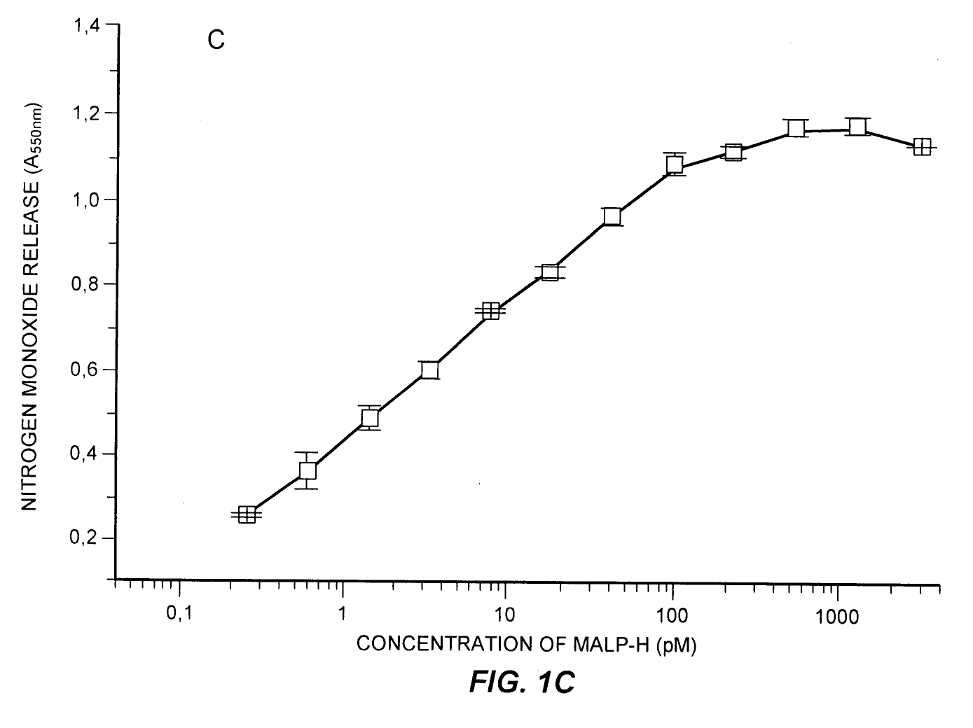

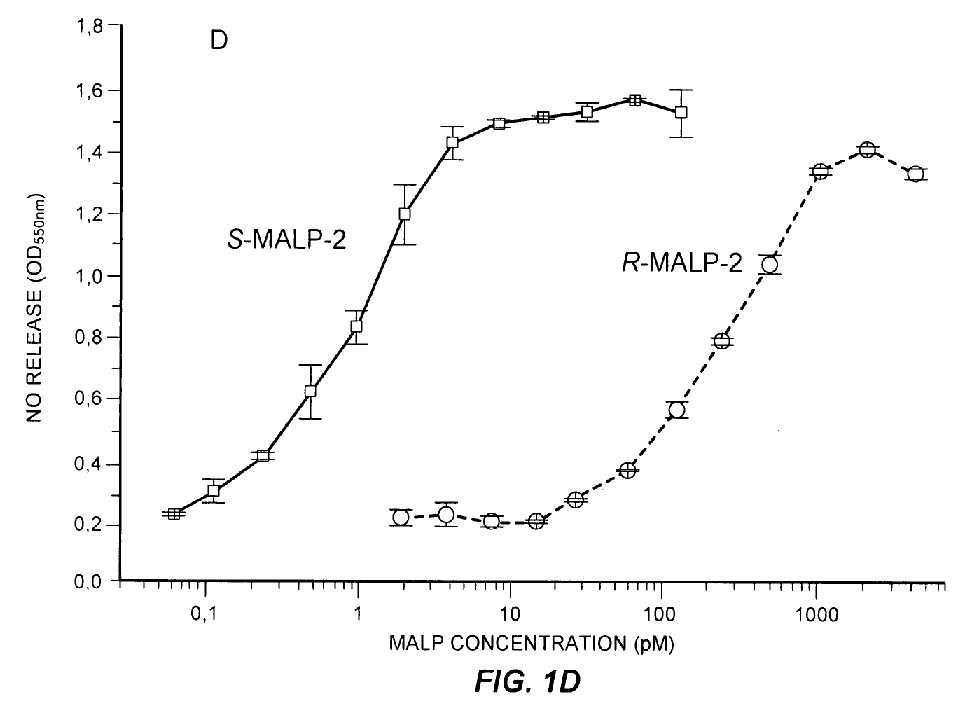

The macrophage-stimulation activity of the following lipopeptides is compared in FIGS. 1A to 1D.

FIG. 1A: MALP-A: S-[2,3-Bispalmitoyloxy-(2RS)-propyl]cysteinyl-SKKKK (SEQ ID NO:6).

FIG. 1B: N-Palmitoyloxy-MALP-A:S-[2,3-Bispalmitoyloxy-(2RS)-propyl]-N-palmitoyl-cysteinyl-SKKKK (SEQ ID NO:6).

FIG. 1C: MALP-H: S-[2,3-Bispalmitoyloxy-(2RS)-propyl]-cysteinyl-GQTNT (SEQ ID NO:5).

FIG. 1D: S-MALP-2: S-[2,3-Bispalmitoyloxy-(2S)-propyl]-cysteinyl-GNNDESNISFKEK (SEQ ID NO:8); and R-MALP-2: S-[2,3-Bispalmitoyloxy-(2R)-propyl]cysteinyl-GNNDESNISFKEK (SEQ ID NO:10).

With the aid of this assay, it is shown that the length or composition of the peptide portion of the lipopeptide has little influence on the macrophage-stimulation activity (see FIGS 1A, 1C and 1D), while the number of fatty acids at the N-terminus (see FIGS. 1A and 1B) and especially the steric configuration in the 2-position of the diacyloxypropyl group is of influence (see FIG. 1D).

### EXAMPLE 2

The model of inflow of granulocytes and macrophages into the peritoneal cavity of the mouse served as an example of the effectiveness of synthetic lipopeptides or liposomes into which such lipopeptides were incorporated. NMRI-outbred mice were used as experimental animals in order to exclude genetic peculiarities.

The racemic lipopeptide MALP-2 was synthesized according to Mühlradt et al., J. Exp. Med., 185:1951-1958 (1997)], the compounds R-MALP-2=S-[2,3-Bispalmitoyloxy-(2R)-propyl]cysteinyl-GNNDESNISFKEK (SEQ ID NO:10) or S-MALP-2=S-[2,3-Bispalmitoyloxy-(2S)-propyl]cysteinyl-GNNDESNISFKEK (SEQ ID NO:8) were synthesized according to reference [Metzger et al., J. Medicinal Chem., 34:1969-1974 (1991)]. MALP-containing liposomes were constructed as follows: The lipids dissolved in chloroform or chloroform/methanol (1+1) (phosphatidyl glycerol, phosphatidyl serine, cholesterol, NBD-PE, molar ratio 1.08:1:0.25:0.005) were pipetted together with the MALP-2 dissolved in 2-propanol/H₂O (1+1) and evaporated using a rotary evaporator. Complete drying of the lipid film occurs overnight in a sterile work bench. Dissolution of the dry lipid film in octyl glucoside (100 mM in 0.05 M Tris-buffered NaCl, pH 7), for 30 minutes at 37° C. in a water bath. Dialysis against a 50-fold volume of NaCl (buffered with 0.1 M Tris, pH 7) at room temperature, replacement of the outside dialysate twice, each time after 24 hours.

The obtained liposome suspension is washed 3× with NaCl (centrifuging for 30 minutes at 47,800 g, 4° C.) and then resuspended in NaCl.

At time 0, the following preparations are injected in a sterile manner and in physiological sodium chloride solution into the peritoncal cavity of the experimental animals. Groups of 6 mice were killed after different times, the peritoneal cavity was washed with 1.2 ml of sterile sodium chloride solution and the number of leukocytes and the leukocyte composition was determined in this cell suspension.

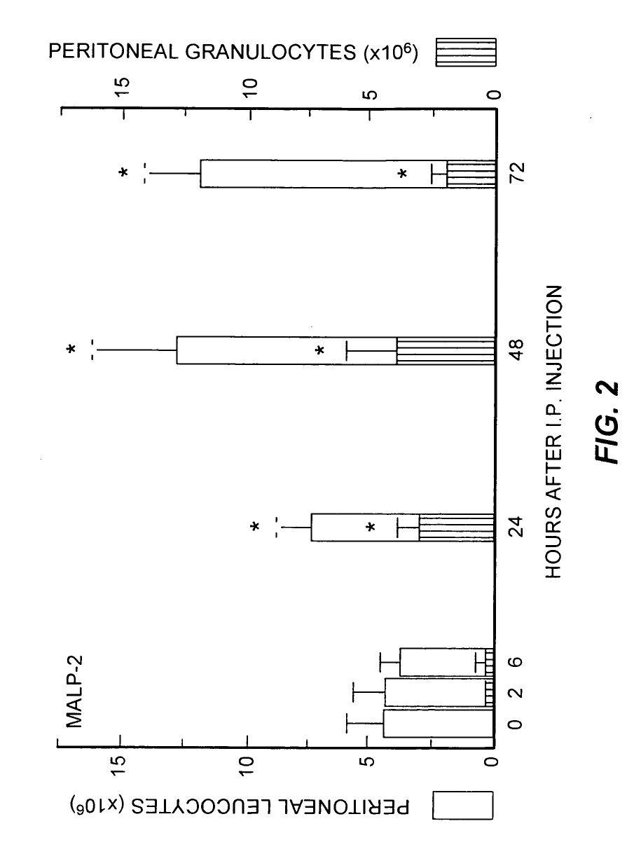

FIG. 2 shows the inflow of the total leukocytes and granulocytes as a reaction to the intraperitoneal injection of 9 μg of racemic MALP-2 into NMRI mice (groups of 6 animals).

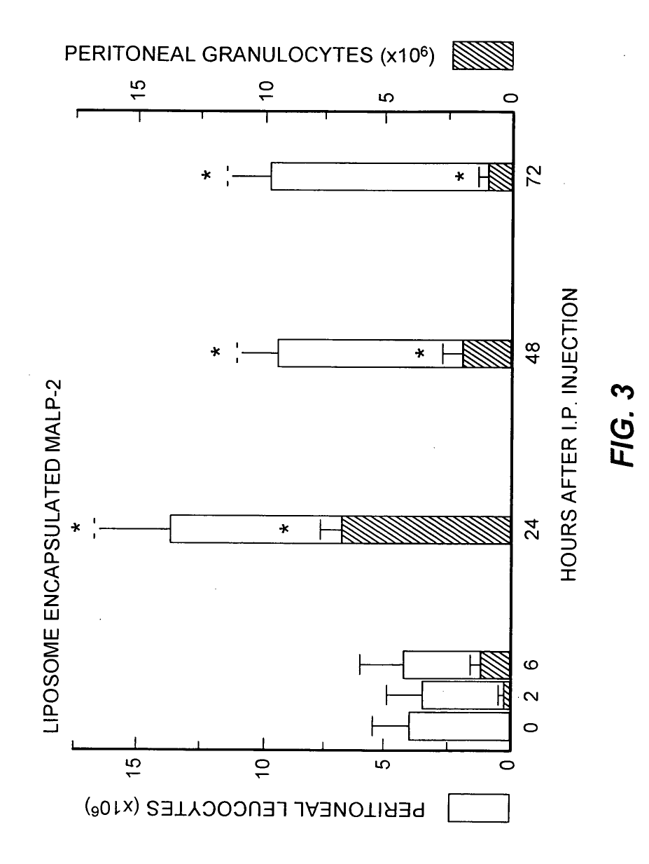

FIG. 3 shows the inflow of the total leukocytes and granulocytes as a reaction to the intraperitoneal injection of 0.2 mg of liposomes, which contained 9 μg of racemic MALP-2, in NMRI mice (groups of 6 animals).

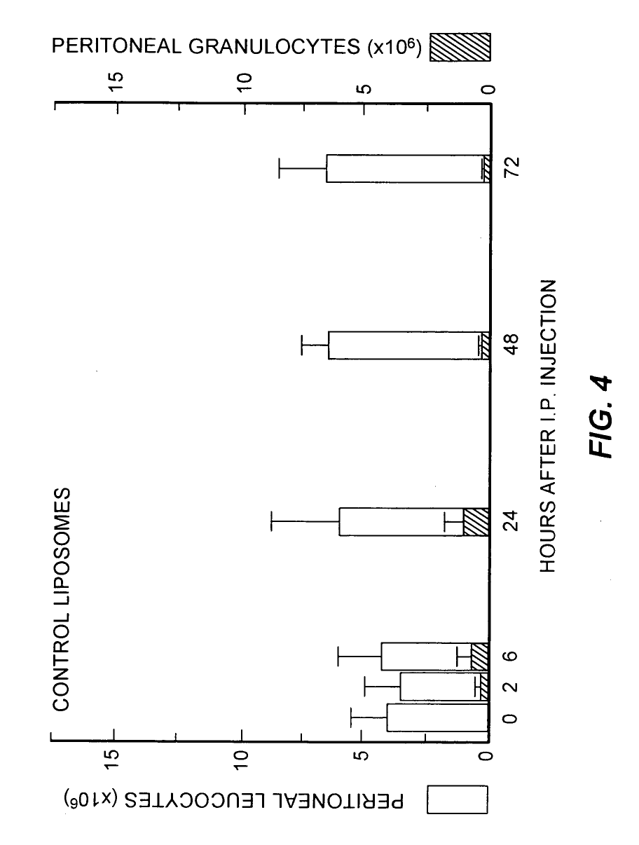

FIG. 4 shows the inflow of total leukocytes and granulocytes as a reaction to the intraperitoneal injection of 0.2 mg of control liposomes which contained no MALP-2, in NMRI mice (groups of 6 animals).

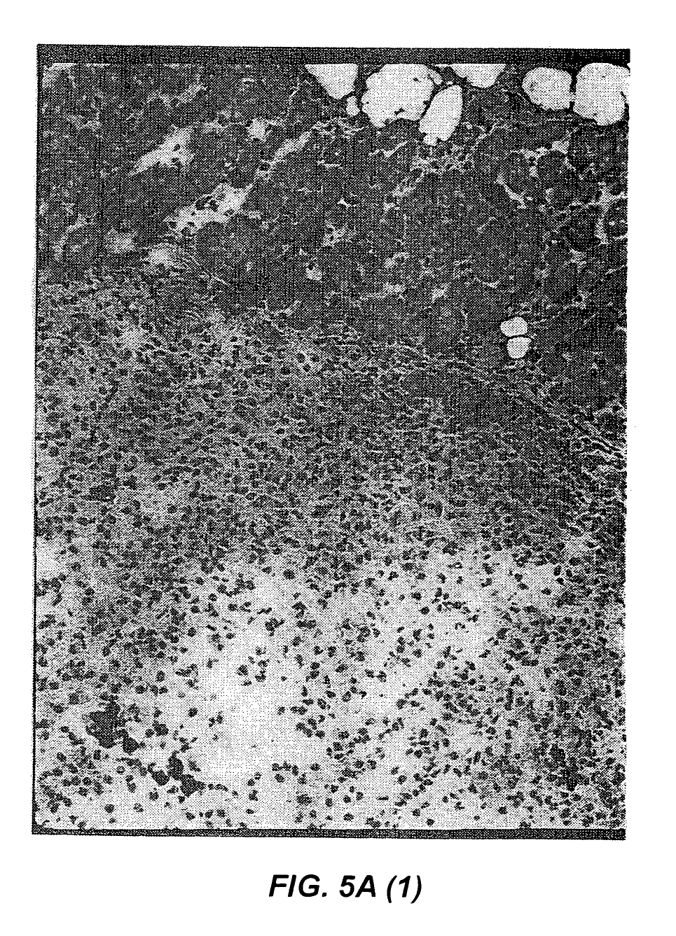

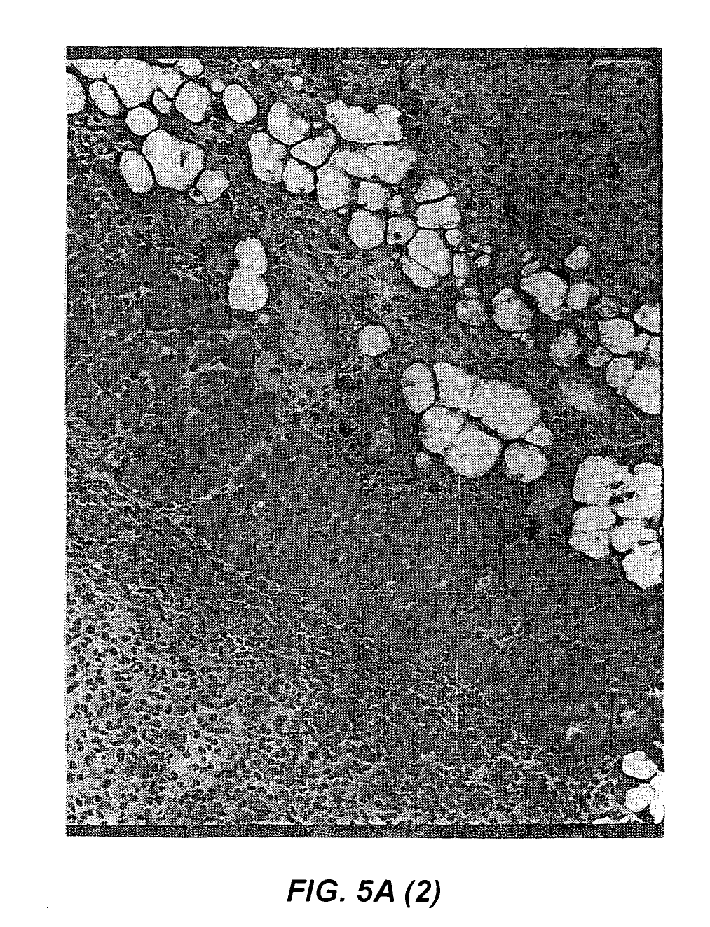

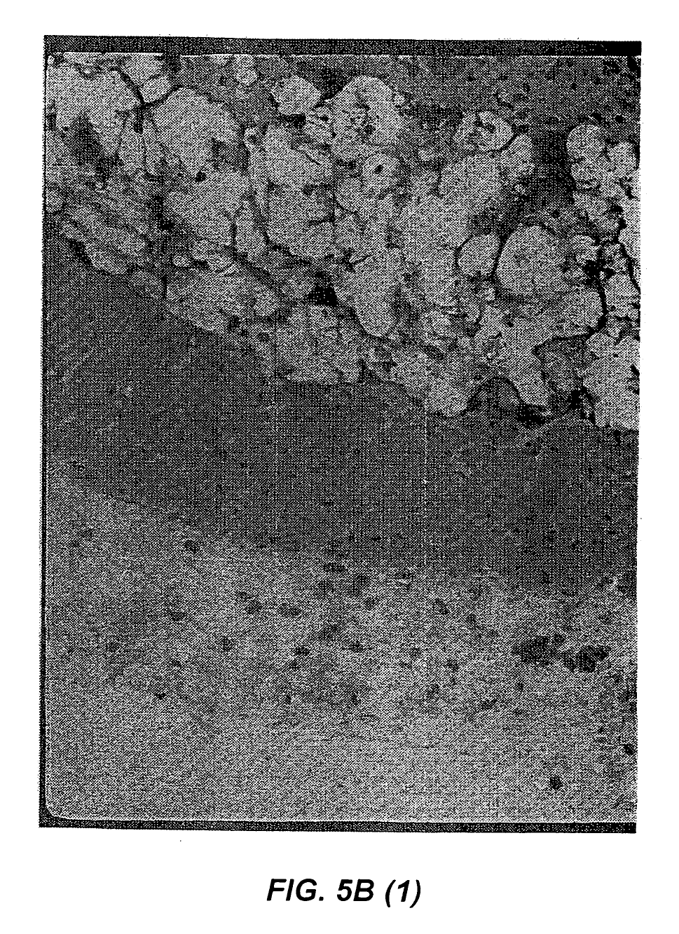

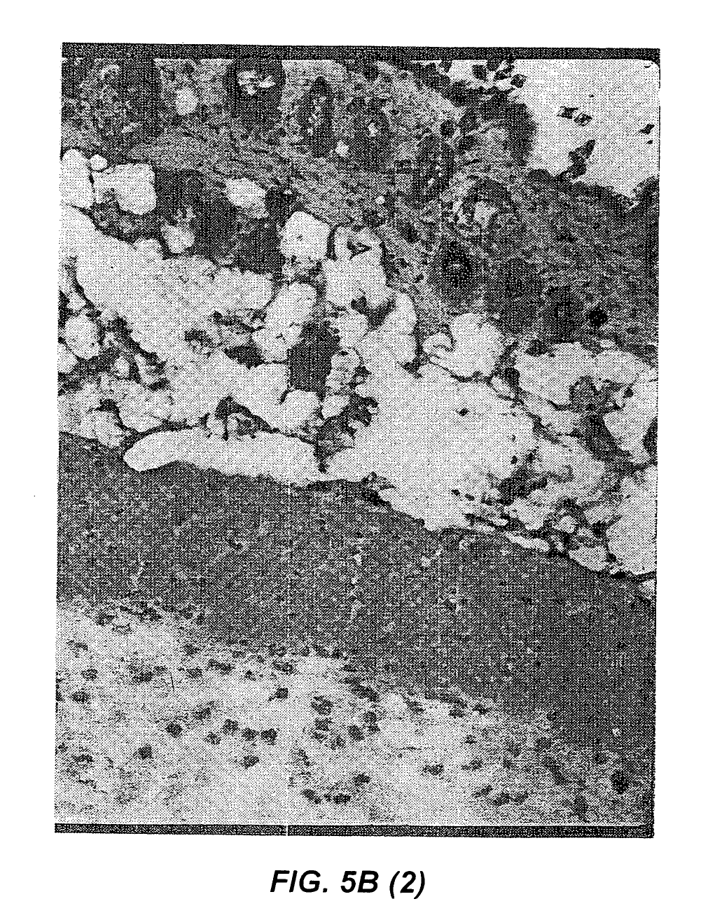

FIG. 5A shows the infiltration of leukocytes in the skin on the back of an NMRI mouse, 3 days after intracutaneous injection of 2 μg of S-MALP-2, which was incorporated into liposomes.

FIG. 5B shows, in comparison: the skin on the back of an untreated mouse.

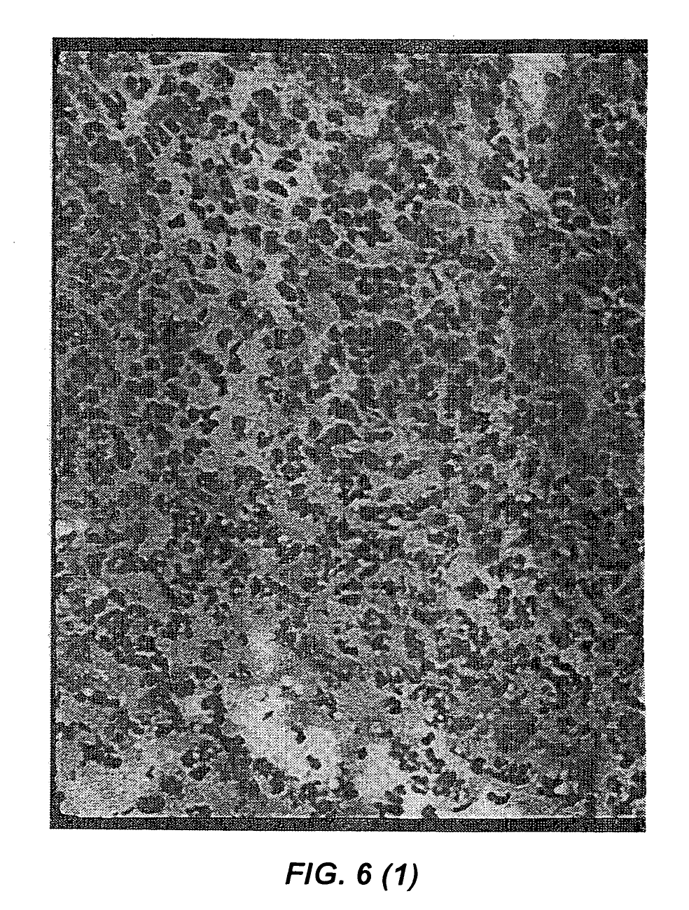

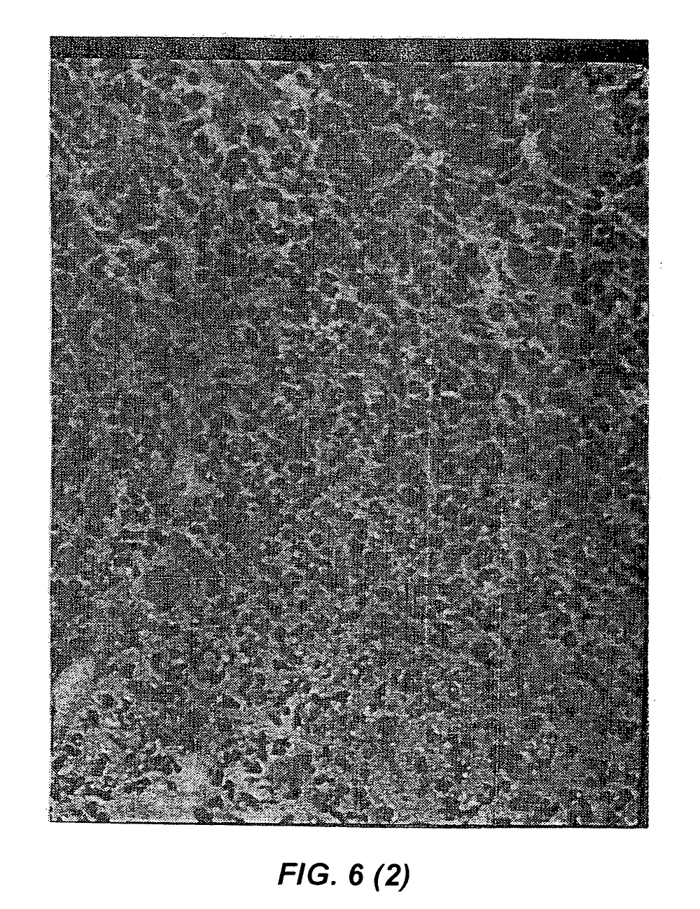

FIG. 6 shows the area of new tissue with new vessels (middle of the picture) formed after MALP injection.

Table 1 shows the essential values, that is, the increase of granulocytes after the injection of the preparations and the later increase of macrophages. In comparison to untreated control animals, an increase of the granulocytes by about 100 times is found, while the macrophages increased two to three times after 3 days.

Significantly increased activities are found in the chemotactically effective chemokines MIP-1α, MIP-2 or KC in the serum of experimental animals measured 2 hours after application of the mycoplasmas or lipopeptide preparations.

### EXAMPLE 3

The importance of the asymmetric C-atom at C2 of the dihydroxypropyl group for the in vivo effect is shown in Table 2. Here, as above, groups of 5 NMRI mice were treated with different amounts of R-MALP-2=S-[2,3-Bispalmitoyloxy-(2R)-propyl]cysteinyl-GNNDESNISFKEK (SEQ ID NO:10) or S-MALP-2=S-[2,3-Bispalmitoyloxy-(2S)-propyl]cysteinyl-GNNDESNISFKEK (SEQ ID NO:8)applied intraperitoneally, and after 3 days the total number as well as the composition of the peritoneal leukocytes were determined. S-MALP-2 is clearly more effective.

### EXAMPLE 4

The following experiment was carried out in order to show the effect of MALP-2 upon intracutaneous application. When 2 μg of free S-MALP or 2 μg of S-MALP incorporated in 0.1 mg of liposomes is applied intracutaneously in the skin of NMRI mice, a clear accumulation of leukocytes is observed at the injection site after 3 days (see FIGS. 5A and 5B) and, after 6 days, new tissue and vessels are formed (FIG. 6). This shows that the preparations are effective in the skin and are able to promote wound healing.

### EXAMPLE 5

Effects of topical application of S-MALP-2 on the infiltration of cells and wound healing in mice.

In order to establish a dose response, two types of experiments were carried out:

1. Determination of infiltration of cells and proliferation by measurement of nucleic acids in skin sections after intracutaneous (ic) injection of various MALP-2 doses. On day 1, the back of C57BLKS/J mice was shaved and hair was removed with Veet. On day 0, the animals were anesthetized with metofane; various doses of MALP-2, dissolved in 30 percent 2-propanol, were injected in volumes of up to 10 μL onto the back (ic). Corresponding volumes of the vehicle without MALP-2 were injected onto the back of the same mice at another location. The animals were killed 10 days later, and punched skin sections (diameter 0.8 cm) were taken from the MALP-2 injection sites and from the control injection sites. The skin biopsies were defatted by extraction with methanol, methanol/chloroform (1+1) and chloroform for 15 minutes and were hydrolyzed in 10 N Hcl overnight at 110° C. The samples were dried, taken up in water and clarified by filtration through a sterile filter (0.2 μm); the nucleic acids were determined by measuring the absorption at 260 nm. Thus, 5 μg of MALP-2 per injection site was found to be optimal. At this dose, the nucleic acid content increased at the MALP-2 injection site by 97±43% in comparison to the corresponding control biopsy (4 animals per group).

2. Cell colonization and proliferation were measured in the skin section (0.8 cm diameter) of wounds which were treated with various amounts of MALP-2. For this experiment, (C57BLKS/J-m+/+Lepr^{db} mice (diabetic) were shaved on the back and hair was removed with Veet. The animals were anesthetized with metofane and their backs were disinfected with Braunol. Then circular skin segments (0.8 cm diameter) were taken from the back of each mouse. The wounds were covered with transparent hydrofilm (Hartmann, Germany); MALP-2 or other vehicles were injected into the wound bed through the film in a volume of 50 μL. After 10 days, the animals were killed; the thin layer of cells, which closes the wound, was sectioned (0.8 mm diameter). The punched samples were hydrolyzed and the nucleic acids determined as before. In this model, too, a dose of 5 μg of MALP-2 per wound was found to be optimal. At this dose, 349±24 μg of nucleic acid per biopsy sample were found in the animals treated with MALP-2 while the value was 142±22 μg in the comparison samples (4 animals per group; p<0.0001).

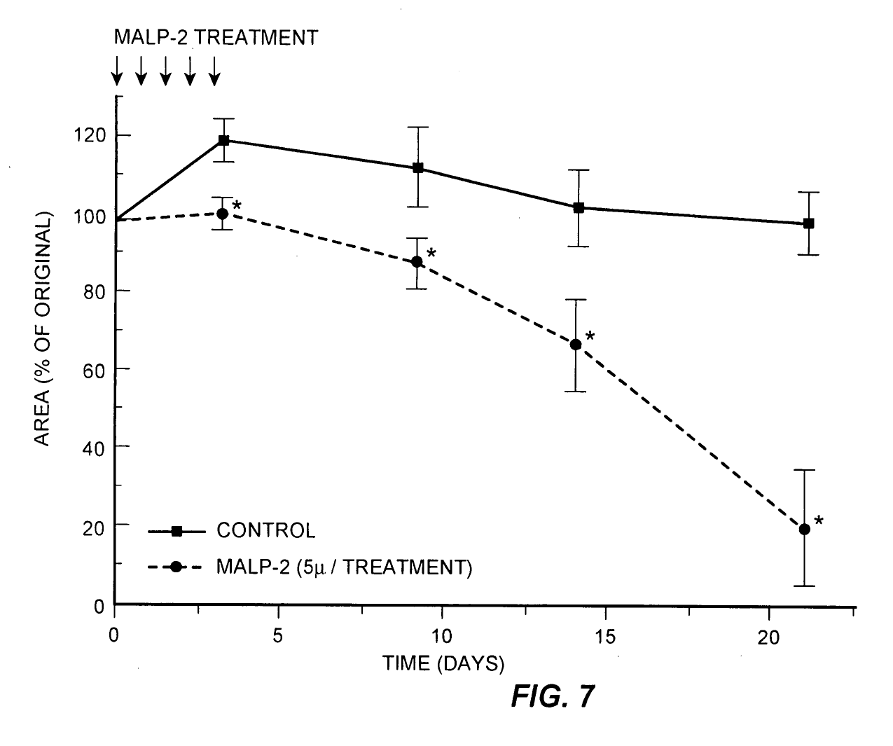

A wound-healing experiment was carried out as follows. On day 1, (diabetic) C57BLKS/J-m+/+-Lepr^{db} mice were shaved on the back. On day 0, the animals were anesthetized with ether and the backs were disinfected with Braunoderm. After that, circular regions (1.3 cm diameter) were cut from the skin of the back of each mouse. The edges of the wound were treated with benzoin tincture; then 100 μL of the MALP-2 preparation or of the vehicles were introduced into each wound (10 mice for each treatment group). The wounds were covered with transparent patches of Tegaderm. Each day, up to day 4, 100 μL of the preparation (content 5 μg of MALP-2) or vehicle preparation was injected into the wound bed through the Tegaderm patches or placed in the wound when the bandage was changed. The mice were kept individually in separate cages. In order to avoid infections, baytril (Bayer) was introduced in the drinking water (50 mg/L of enrofloxacin). The bandages were changed once a week under ether anesthesia, and the wound edges were recorded on microscope slides. The areas shown were determined with a standard CCD camera using Scionlmage software. The wounds were checked visually for infections; liquid samples from obviously infected wounds were tested on blood agar plates. Mice with infected wounds were removed from further analyses. The data are given in FIG. 7 and show a significant acceleration of wound healing in the case of the animals treated with S-MALP-2.

### EXAMPLE 6

### Increase of the Survival Rate of Mice in a Peritonitis Model by Pretreatment with MALP-2

“Colon ascendens stent peritonitis” is a new model for abdominal sepsis, which may follow surgical intervention [Zantl et al., Infekt. Immun., 66:2300-2309 (1998)]. The following experiments were carried out in order to evaluate the ability of MALP-2 to increase the survival rate in this model. Groups of 12 to 18 C57BL/6 mice were injected intraperitoneally with 2 μg of S-MALP-2 in 200 μL of physiological sodium chloride solution or with the same volume of physiological sodium chloride solution either 2 or 4 days before surgical intervention. The surgical intervention was carried out under ether anesthesia. The abdomen was disinfected with 70% ethanol and then opened by section in the middle line (1 cm). A venous catheter (16 gauge) was inserted into the lumen of the descending colon and secured with two stitches using Ethilon thread (7/0). Then the inner needle of the stent was removed, the catheter was shortened to a length of a few millimeters and a small drop of feces was pressed out from the catheter. The peritoneal cavity was rinsed with 0.5 Ml of sodium chloride solution before closing the abdomen. Among the mice with the control, sodium chloride solution (18 per group), none lived longer than 90 h. Among those who were treated two days before the surgical intervention with MALP-2, 4 out of 12 survived for 150 h or longer, while among the animals who were treated with MALP-2 four days before the surgical intervention, 6 animals out of 12 survived for 120 hours and longer. The difference of survival rate was statistically significant (P<0.001 according to the log-rank test).

### REFERENCED STATE OF THE ART

[1] Quentmeier et al. in Infect. Immum., 58 (1990) 1273-1280

[2] Mühlradt & Frisch in Infect. Immun., 62 (1994) 3801-3807

[3] Mühlradt & Schade in Infect. Immun., 59 (1991) 3969-3974

[4] Metzger et al. in Int. J. Pep. Protein Res., 38 (1991) 545-554

[5] Metzger et al. in J. Pept. Sci., 3 (1995) 184-190

[6] Mühlradt et al. in J. Exp. Med., 185 (1997) 1951-1958

[7] Metzger et al. in J. Medicinal Chem., 34 (1991) 1969-1974

[8] Hausschild et al. in FEMS Immunol. Med. Microbiol., 8 (1994) 77

[9] Zantl et al. in Infekt. Immun., 66 (1998) 2300-2309

[10] Mühlradt et al. in Infect. Immun., 66 (1998) 4804

[11] Kaufinann et al., in Infect. Immun., 67 (1999) 6303

[12] Deiters & Mühlradt in Infect. Immun., 67 (1999) 3390

[13] Takeuchi et al. in J. Immunol., 164 (2000) 554

TABLE 1
Leukocyte inflow as a sequel to intraperitoneal injection of racemic MALP-2 after 72 hours

| | Peritoneal | Monocytes + | | | | | |
|---|---|---|---|---|---|---|---|
| | cells | macrophages | | Lymphocytes | | Granulocytes | |
| Treatment | (×10⁶) | (×10⁶) | (%) | (×10⁶) | (%) | (×10⁶) | (%) |
| MALP-2 (9 μg) | 11.2 ± 3.1^{b} | 5 ± 1.2^{b} | 47.4 ± 15.2 | 3.5 ± 1.2 | 32 ± 11^{b} | 2.6 ± 2.7 | 20.2 ± 13.2^{b} |
| liposome- | 8.5 ± 1.2^{c} | 4.9 ± 0.9^{c} | 57.6 ± 8.1^{c} | 2.9 ± 1 | 33.4 ± 9^{c} | 0.8 ± 0.2^{c} | 9.7 ± 2.2^{c} |
| encapsulated | | | | | | | |
| MALP-2 (9 μg) | | | | | | | |
| control | 6.4 ± 1.9 | 2.9 ± 1.2 | 44.5 ± 6.8 | 3.2 ± 0.9 | 51.8 ± 6.7 | 0.2 ± 0.08 | 2.9 ± 1.4 |
| liposomes | | | | | | | |
| NaCl (0.9%) | 5.8 ± 0.5 | 2.8 ± 0/4 | 47.9 ± 1.7 | 2.8 ± 0.2 | 48.8 ± 1 | 0.05 ± 0.03 | 0.9 ± 0.6 |

Groups of 6 animals were used.
^{b}Significant difference (P < 0.05) in comparison to control animals treated with sodium chloride (according to the Student t-test).
^{c}Significant difference (P < 0.05) in comparison to the animals which received the control liposomes (according to the Student t-test).

TABLE 2
Leukocyte inflow 72 hours after intraperitoneal injection of R-MALP-2 or S-MALP-2

| | | PEC | macrophages | | lymphocytes | | neutrophils | |
|---|---|---|---|---|---|---|---|---|
| | | (×10⁶) | (×10⁶) | % | (×10⁶) | % | (×10⁶) | % |
| control | A | 5.4 | 2.9 | 54 | 2.2 | 40.7 | 0 | 0 |
| | B | 6.4 | 3.8 | 58.6 | 2.4 | 38.1 | 0.19 | 0.3 |
| | C | 7.95 | 4.1 | 52.6 | 3.4 | 43.1 | 0.24 | 0.3 |
| | D | 7.5 | 2.9 | 38.5 | 4.4 | 57.9 | 0 | 0 |
| | E | 5.1 | 2.7 | 53.4 | 2.2 | 44 | 0 | 0 |
| | φ | 6.5 ± 1.3 | 3.3 ± 0.6 | 51.4 ± 7.6 | 2.9 ± 0.9 | 44.8 ± 7.7 | 0.09 ± 0.1 | 0.12 ± 0.16 |
| R-MALP | A | 11.1 | 4.5 | 40.1 | 6.3 | 56.5 | 0.56 | 0.5 |
| (10 μg) | B | 4.05 | 2.1 | 52.4 | 1.8 | 45.2 | 0.32 | 0.8 |
| | C | 11.4 | 6 | 53 | 5 | 44.2 | 0.34 | 0.3 |
| | D | 7.65 | 4 | 52.6 | 3.4 | 44.4 | 0 | 0 |
| | E | 9 | 4.6 | 51.3 | 4.1 | 45.6 | 0.27 | 0.3 |
| | φ | 8.64 ± 3 | 4.2 ± 1.4 | 49.9 ± 5.5 | 4.1 ± 1.7 | 47.2 ± 5.2 | 0.3 ± 0.2 | 0.4 ± 0.3 |
| S-MALP | A | 9.1 | 4.5 | 49.9 | 4.2 | 46.3 | 0.46 | 0.5 |
| (1 μg) | B | 10.8 | 4.7 | 43.8 | 5.2 | 48.3 | 3 | 2.8 |
| | C | 9.35 | 5.3 | 56.9 | 2.6 | 27.8 | 12 | 12.8 |
| | D | 13.7 | 8.4 | 61.6 | 4 | 29.4 | 10.1 | 7.4 |
| | E | 7.9 | 4.6 | 58.5 | 2.8 | 34.9 | 2.8 | 3.5 |
| | φ | 10.2 ± 2.2^{a} | 5.5 ± 1.7^{a} | 54.1 ± 7.2 | 3.8 ± 1.1 | 37.3 ± 9.5 | 14.8 ± 17.9^{a,b} | 5.4 ± 4.8^{a,b} |
| S-MALP | A | 14.4 | 6.3 | 44.1 | 5.1 | 35.2 | 25.9 | 18 |
| (5 μg) | B | 15.2 | 5.6 | 37.2 | 5.4 | 35.5 | 41 | 27 |
| | C | 17.3 | 6.1 | 34.8 | 10 | 58.1 | 8.1 | 4.7 |
| | D | 13.1 | 5.4 | 41.5 | 2.8 | 21.5 | 45.5 | 34.7 |
| | E | 12.5 | 6.3 | 50.7 | 4.2 | 33.4 | 17.3 | 13.8 |
| | φ | 14.5 ± 1.9^{a,b} | 5.9 ± 0.4^{a,b} | 41.7 ± 6.2 | 5.5 ± 2.7 | 36.7 ± 13.3 | 27.6 ± 15.7^{a-,b} | 19.6 ± 11.−6^{a,b} |

^{a}Significant differences in comparison to the control animals (no injection).
^{b}Significant differences to the animals treated with R-MALP (10 μg).

1. A method of treating a wound in an animal or human comprising administering to said animal or human a pharmaceutical composition comprising a lipopeptide or lipoprotein with the following general structure: 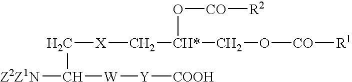
wherein
R¹ and R² stand for C₇₋₂₅-alkyl, C₇₋₂₅-alkenyl or C₇₋₂₅-alkinyl,
X is S, O, or CH₂,
Z¹ and Z² stand for H or methyl,
W stands for CO or S(O)ₙ (where n=1 or 2) and
Y stands for a physiologically compatible amino acid sequence consisting of 1 to 25 amino acid residues and the asymmetric carbon atom marked with * denotes an asymmetric carbon atom and has the absolute configuration R when X═S (sulfur).

2. The method of claim 1, wherein Y comprises an amino acid sequence consisting of 1 to 25 amino acids.

3. The method of claim 1, wherein Y comprises an amino acid sequence which is selected from the group consisting of:
(i) amino acid sequence, which does not have an adverse influence on the water solubility of the lipopeptide or lipoprotein;

| | | |
|---|---|---|
| (ii) | GQTNT; | (SEQ ID NO:1) |
| (iii) | SKKKK; | (SEQ ID NO:2) |
| (iv) | GNNDESNISFKEK; | (SEQ ID NO:3) |
| (v) | GQTDNNSSQSQQPGSGTTNT; | (SEQ ID NO:4) |

or a fragment or variant of the amino acid sequences in (ii), (iii), (iv) and (v) wherein said fragment or variant has macrophage stimulating activity.

4. The method of claim 1 wherein the C₇₋₂₅-alkyl, C₇₋₂₅-alkenyl, or C₇₋₂₅-alkinyl is a C₁₅-alkyl, C₁₅-alkenyl or C₁₅-alkinyl, respectively.

5. The method of claim 1 wherein the double bond(s) in the C₇₋₂₅-alkenyl group has(have) the cis-configuration.

6. A method of treating a wound in an animal or human comprising administering to an animal or human a physiologically compatible lipopeptide or lipoprotein which carries at the N-terminal a dihydroxypropyl cysteine group with two, fatty acids bonded via ester bonds.

7. The method of claim 1 wherein said lipopeptide or lipoprotein is obtained from a mycoplasma clone.

8. The method of claim 7, wherein said lipopeptide or lipoprotein is obtained from a Mycoplasma fermantans clone.

9. The method of claim 1 wherein said the lipopeptide or lipoprotein is water-soluble or amphoteric.

10. The method of claim 1 wherein said lipopeptide or lipoprotein selected from the group consists of:
(i) S-[2,3-bispalmitoyloxy-(2RS)-propyl]cysteinyl-GQTNT (SEQ ID NO:5)
(ii) S-[2,3-bispalmitoyloxy-(2RS)-propyl]cysteinyl-SKKKK (SEQ ID NO:6)
(iii) S-[2,3-bispalmitoyloxy-(2RS)-propyl]cysteinyl-

| | |
|---|---|
| GNNDESNISFKEK | (SEQ ID NO:7) |

(iv) S-[2,3-bispalmitoyloxy-(2S)-propyl]cysteinyl-

| | |
|---|---|
| GNNDESNISFKEK | (SEQ ID NO:8) |

(v) S-[2,3-bispalmitoyloxypropyl]cysteinyl-

| | |
|---|---|
| GQTDNNSSQSQQPGSGTTNT | (SEQ ID NO:9) |
| and | |

(vi) S-[2,3-bispalmitoyloxy-(2R)-propyl]cysteinyl-

| | |
|---|---|
| GNNDESNISFKEK. | (SEQ ID NO:10) |

11. The method of claim 1 wherein said lipopeptide or lipoprotein is in the form of a solution for epicutaneous application, an injection solution, a salve, a lotion, an aqueous suspension, a plaster impregnated or coated with said lipopeptide or lipoprotein, encapsulated in liposomes, or coupled to biodegradable carrier polymers.

12. The method of claim 1 wherein said wounds are wounds after injury or surgical intervention, chronically infected wounds, burn wounds, chronic ulcers or Ulcus venosum or wounds of patients who are corpulent or diabetic or are subjected to radiation or chemotherapy.
